(19) United States
(12) Patent Application Publication
(10) Pub. No.: US 2005/0048088 A1
(43) Pub. Date: Mar. 3, 2005

(54) Compositions comprising at least two nanoemulsions

(76) Inventors: Fred Zulli, Kuttigen (CH); Franz Suter, Dottingen (CH); Christina Liechti, Aarau (CH); Esther Belser Gisi, Buchs (CH)

Correspondence Address: Paul J. Maginot;Suite 3000, 111 Monument Circle, Indianapolis, IN, 46204-5115, US

(21) Appl. No.: 10/922,196

(22) Filed: Aug. 19, 2004

(30) Foreign Application Priority Data
Aug. 28, 2003 (CH) 1466/03

(51) Int. Cl. A61K 38/44; A61K 9/00

(52) U.S. Cl. 424/401; 424/94.4

(57) ABSTRACT

The stable compositions of the present inventions comprise at least two different nanoemulsions stabilized by lecithin each of which containing a liquid lipid, at least two of said lipids are incompatible to each other. Particularly said compositions comprise as incompatible lipids tocopherol and Coenzyme Q10. Said composition are useful in cosmetics, in cell cultures and in nutrient compliments. Processes are described for preparing such compositions form lipids which are solid at room temperature.

### FIELD OF THE INVENTION

This invention relates to compositions comprising at least two nanoemulsions, d to methods of preparing them, and to their.

Nanoemulsions, alternatively called nanoparticles, are composed of oil particles, the surfaces of which are occupied by an amphoteric emulsifier in aqueous dispersions. Suitable emulsifiers are lecithin or other emulsifiers, such as e.g. poloxamers (international generic name for copolymers of polyethylenglycols and polypropylenglycols) or sodium cholate. Preferably, the occupation of the surface of the oil particles is in the form of a monolayer. In particular, the nanoemulsions comprise per part by weight of oil more than 0.4 parts by weight, and preferably more than 0.45 to 1.0 parts by weight, of said amphoteric emulsifier. Usually, the diameter of said oil particles is from 20 to 1000 nm.

Usually, the nanoemulsions have a negative zeta potential, and preferably between −10 mV und −50 mV, and more particularly between −30 mV and −40 mV. However, for special applications nanoemulsions having a positive zeta potential may be of advantage. Such cationic nanoemulsions can e.g. be obtained by addition of a C8- to C22-alkylamide.

Nanoemulsions are at will miscible with water. They are very stable and can even be autoclaved.

Nanoemulsions can be prepared by mixing lipids, e.g. triglycerides, in an aqueous phase, with lecithin in a high-pressure homogenize (e.g. a Microfluidizer®). The preparation of such nanoemulsions is, e.g., described in EP-B1-0 406 162.

Nanoemulsions are e.g. used in cosmetics for transporting active components into deeper skin layers. In cell cultures nanoemulsions can be used for complementing aqueous mediums with lipophilic substances (US-B1-6 265 180), e.g. for improving the production of antibodies. Furthermore, nanoemulsions are used for determining the biocompatibility/toxicity of lipid in cell culture tests (US-B1-6 265 180). Nanoemulsions are also used in nutrient compliments for increasing the bioavailability of lipophilic substances, such as e.g. Coenzyme Q10, in aqueous products.

Usually, liquid oils, or mixtures of various lipophilic substances or oils, are used for preparing nanoemulsions. However, it may happen that the used lipophilic substances are not compatible with each other. Therefore, in this case, they cannot processed together for preparing nanoemulsions.

In particular, it is e.g. not possible to process tocopherol and Coenzyme Q10 together in nanoemulsions. The two lipophilic substances react with each other by electron transfer processes, and the solution changes its color to brown. Moreover, usual emulsions, such as creams, cannot be prepared because the incompatible substances would react in the cream. If the two substances are individually processed into emulsions and the emulsions thereafter mixed, the incompatible lipids nevertheless react with each other since the oil droplets will mix little by little.

As said above, usually liquid oils are used for preparing nanoemulsions. These oils may be several components. Thereby, lipophilic substances may be dissolved in the oils as well, the obtained mixture being liquid at room temperature.

The preparation of so-called solid-lipid-nanoparticles (SLN) was also described. These dispersions comprise at room temperature solid lipid particles which are dispersed in water by means of an emulgator, e.g. lecithin. These dispersions are prepared by melting the lipid, which is then processed at high temperatures to a nanoemulsion. Upon cooling solid lipid particles are again formed. These particles are suitable as “Controlled-release” vehicles for active components which are poorly soluble in water and are dissolved in the lipid particles, and which then slowly diffuse into the aqueous phase (US-B1-6 207 178). One disadvantage of these solid-lipid-nanoparticles is that the lipophilic substances, which are solid matter, have only a very poor bioavailability, this as well in cosmetics, in cell cultures and as nutrient compliments. Another disadvantage of these SLN dispersions is that the lipids are to be heated to their melting points and are to be processed at said temperatures. At an industrial scale this process is expensive, and the lipids and/or active components may be destroyed.

An interesting substance having many uses in cosmetics, cell cultures and nutrient components is Coenzyme Q10 (Ubiquinone). This lipid is solid at room temperature and melts at approximately 50° C. US-B1-6 197 349 describes the preparation of a nanoemulsion comprising Coenzyme Q10[CoQ10] in the form of a supercooled melt. In this case, the molten CoQ10 remains liquid in the nanoemulsion, contrary to SLP nanodispersions. One disadvantage of this composition is that the preparation of the nanoemulsion both the aqueous phase and the lipid phase are to be heated to 70° C. Such high temperatures are harmful for the CoQ10 and for other active compounds which are included. Another disadvantage is that the oily phase which consists only of CoQ10 is not suitable for the preparation of very small oil droplets and high concentrations of CoQ10. The examples of US-B1-6 197 349 exclusively describe nanoemulsion having particle sizes of more than 67 nm. The nanoemulsions containing more than 3 percent by weight of CoQ10 are even larger than 100 nm. However, the bioavailability of small particles is much better, particularly if said particles are of the same size as viruses (6 to 50 nm).

### OBJECTS OF THE INVENTION

It is the primary object of the invention to solve the above mentioned problems by creating a composition containing incompatible lipids fully separated in different oil droplets which can be mixed without any reaction of the oil droplets.

Another object is to create such compositions having a good stability and a good bioavailability.

The forgoing and further objects, advantages and features will be apparent from the following specification.

### SUMMARY OF THE INVENTION

To meet theses and other objects, the invention provides the following compositions, the following methods, and the following uses of such compositions:
a composition comprising at least two different nanoemulsions stabilized by lecithin each of which containing a liquid lipid, at least two of said lipids being incompatible with each other.
a process for preparing a composition comprising at least two different nanoemulsions stabilized by lecithin each of which containing a liquid lipid, at least two of said lipids being incompatible, and at least one of said lipids being in solid form at room temperature, said process comprising the steps of:
dissolving in oil or a mixture of oil and an organic solvent, separately from the other lipids, at least one of the incompatible solid lipids at a temperature at which it is soluble in said oil or said mixture, respectively;
processing each of the solutions of lipids separately into an individual nanoemulsion by means of high pressure homogenization;
cooling each of said nanoemulsions down to room temperature thereby creating a stable supersaturated solution of said solid lipid; and;
combining the individual solutions to form said composition.
the use of a composition comprising at least two different nanoemulsions stabilized by lecithin each of which containing a liquid lipid, at least two of said lipids being incompatible with each other, in cosmetic preparations, in cell cultures and/or in nutrient compliments.

### DESCRIPTION OF THE PREFERRED EMBODIMENTS

Surprisingly, the above mentioned composition, comprising so-called “multiple nanoemulsions”, is very stable and can be stored at room temperature for at least 6 months, preferably for at least 2 years, and in a refrigerator up to 3 years, without reaction of the incompatible lipids.

For use in cell culture tests the compositions can either be autoclaved, or alternatively, if the droplet size is small (0.1 μm), be sterilized. This makes it possible to offer and apply different incompatible lipids in the same preparation. This dramatically simplifies the use of said compositions, not only in cell cultures, but also in cosmetic and in nutrient compliments.

Furthermore, it is possible to prepare transparent mixtures using particles which are smaller than 80 nm. Transparent nanoemulsions allow the formulation of aesthetically appealing transparent cosmetic hydrogels. When working with cell cultures transparent nanoemulsions facilitate the visual control. Moreover, the bioavailability of very small oil droplets in the range of 20 to 80 nm on the skin, in cell cultures and in nutrient compliments is strongly increased.

If one of the lipophilic compounds to be processed is solid at room temperature it is first to be dissolved in a lipophilic carrier.

The problem of providing an improved form of administration of solid lipophilic compounds, particularly of the lipophilic active component Coenzyme Q10, is solved by providing a nanoemulsion which comprises as liquid oil phase or oil phases, respectively, a solution or solutions, respectively, of said lipophilic compound or compounds, respectively, in a suitable oil. Such carrier oils should be suitable for preparing ultra-small oil droplets by high pressure homogenization. Unfortunately, many interesting active compounds, such as said Coenzyme Q10, have only a poor solubility at room temperature. Often, the solubility of such active components can significantly increased by warming said carrier oils and/or additionally diluting them with alcohol or other solvents. But on cooling said solutions, prepared at higher temperatures, down to room temperature the active compounds gradually recrystallize out. However, if said warmed solutions are directly processed to nanoemulsions, to one's surprise, very stable supersaturated solutions are obtained. If stored at 4° C., such supersaturated solutions remain stable for several years.

For characterizing the state of aggregation of said Coenzyme Q10 in nanoemulsions, the preparations can be tested by means of Differential Scanning Calorimetry DSC. If the Coenzyme Q10 is present in liquid form, no melting process can be determined. In most cases, recrystallization of the lipids form supersaturated solutions will destroy the nanoemulsions. At first, the particles increase in size, finally the nanoemulsion breaks down, and the lipids preticipate.

The supersaturated nanoemulsions described above show a number of advantages as compared with nanoemulsions consisting of a supercooled melt:
1. The lipids are not to be heated beyond the melting point.
2. The aqueous phase is not to be heated.
3. By this, the preparing process is much simpler and less expensive.
4. Due to the free choice of a suitable carrier oil, much smaller particles (e.g. smaller than 67 nm) having a better bioavailability can be produced.
5. Due to the free choice of a carrier oil higher concentrations of the lipid can be processed into the nanoemulsion without increasing the particle size to much.

Possible compositions of such multiple nanoemulsions, and multiple nanoemulsions comprising supersaturated solutions, are explained in the following examples.

All numerals given below are percents by weight. The indication of the ingredients is made according to the INCI (International Nomenclature of Cosmetics Ingredients) nomenclature.

### EXAMPLES

### Example 1

Transparent Double Nanoemulsion Comprising Coenzyme Q10 and Alpha-tocopherol

Nanoemulsion 1—Composition

| | |
|---|---|
| Lecithin | 3.5% |
| Tocopheryl Acetate | 3% |
| Caprylic/Capric Triglyceride | 3% |
| Ubiquinone (Coenzyme Q10) | 1% |
| Diisopropyl Adipate | 1% |
| Alcohol | 12% |
| Glycerin | 20% |
| Aqua | 59% |

Preparation of 1 kg Nanoemulsion 1

10 g of Coenzyme Q10 (ubiquinone) are) were dissolved at 40° C. in 30 g of tocopheryl acetate and 30 g of caprylic/capric triglyceride. 35 g of lecithin were dissolved in 120 g of alcohol and added with stirring to a mixture of 590 g of water and 200 g of glycerin. The two phases were combined and then homogenized five times at 1200 bar (1.2·10⁸ Pa) using a high pressure homgenizer of Microfluidics Corp (MT 110®). Determination of the particle size by means of photon correlation spectroscopy (Autosizer 3C®) shows a mean particle size of 63.4 nm.

Nanoemulsion 2—Composition

| | |
|---|---|
| Lecithin | 5% |
| Caprylic/Capric Triglyceride | 4% |
| Tocopherol | 1% |
| Alcohol | 15% |
| Glycerin | 20% |
| Aqua | 55% |

Preparation of 1 kg of Nanoemulsion 2

Nanoemulsion 2 was prepared the same way as Nanoemulsion 1. The particle size was 41.6 nm. Nanoemulsions 1 and 2 were mixed at a ratio of 1:1. The obtained transparent double nanoemulsion comprising Coenzyme Q10 (ubiquinone) and tocopherol shows for at least 15 months no decoloration at 4° C., at room temperature and at 37° C., and the original particle size of 68.6 nm remains stable. Thus, the double nanoemulsion has an excellent storage stability.

### Example 2

Transparent Double Nanoemulsion Comprising an Supersaturated Solution of Coenzyme Q10

Nanoemulsion 3—Composition

| | |
|---|---|
| Lecithin | 3% |
| Vegetable Oil | 4% |
| Ubiquinone (Coenzyme Q10) | 1% |
| Alcohol | 20% |
| Glycerin | 20% |
| Aqua | 52% |

Preparation of 1 kg of Nanoemulsion 3

30 g of lecithin were dissolved in 100 g of alcohol and added with stirring to 520 g of water and 200 g of glycerin. 10 g of Coenzyme Q10 (ubichinone) [CoQ10] were dissolved at 40° C. in 40 g of vegetable oil and 100 g of alcohol (ethanol). When this CoQ10 solution was again cooled to room temperature (25° C.) most of the CoQ10 recrystallizes out from the solution after some hours. Therefore, the CoQ10 solution, having a temperature of 40° C., was added to the lecithin/alcohol/glycerin/water mixture and then homogenized six times at 1200 bar (1.2·10⁸ Pa) using a high pressure homgenizer of Microfluidics Corp (MT 110®), Thereby, the alcohol was homogeneously distributed in the nanoemulsion. An orange transparent nanoemulsion was obtained. Determination of the particle size by means of photon correlation spectroscopy (Autosizer 3C®) shows a mean particle size of 45.0 nm.

Samples of this nanoemulsion can be incubated at 4° C., at room temperature, and at 37° C. for one year, without any essential change of the particle size. Measurement with a differential calorimeter (Perkin Elmer) does not show a phase transition for the Coenzyme Q10 in the nanoemulsion. This means that Coenzyme Q10 in the nanoemulsion is in dissolved form. Since at 4° C. and at room temperature the limit of solubility of Coenzyme Q10 in vegetable oil is clearly exceeded, the preparation in question is a stable nanoemulsion of a supersaturated solution.

Nanoemulsions 3 and 2 were mixed at a ratio of 1:1. The obtained transparent double nanoemulsion comprising a supersaturated solution of Coenzyme Q10 (ubiquinone) and tocopherol shows for at least 20 months no decoloration at 4° C., at room temperature, and at 37° C., and the original particle size of 65.0 nm remains stable. Thus, the double nanoemulsion has an excellent storage stability.

### Example 3

Double Nanoemulsion Comprising Various Vitamins

Nanoemulsion 4—Composition

| | |
|---|---|
| Lecithin | 5% |
| Tocopheryl Acetate | 2% |
| Caprylic/Capric Triglyceride | 2% |
| Ubiquinone (Coenzyme Q10) | 0.5% |
| Retinyl Palmitate | 0.5% |
| Alcohol | 15% |
| Glycerin | 20% |
| Aqua | 55% |

Preparation of 1 kg of Nanoemulsion 4:

Nanoemulsion 4 was prepared the same way as Nanoemulsion 1. The particle size was 56.3 nm. Nanoemulsion 5—Composition

| | |
|---|---|
| Lecithin | 3% |
| Caprylic/Capric Triglyceride | 3% |
| Tocopheryl Acetate | 2.5% |
| Borago Officinalis Seed Oil | 1% |
| Tocopherol | 0.4% |
| Ascorbyl Tetraisopalmitate | 0.1% |
| Alcohol | 15% |
| Glycerin | 20% |
| Aqua | 55% |

Preparation of 1 kg of Nanoemulsion 5

Nanoemulsion 4 was prepared the same way as Nanoemulsion 1. The particle size was 61.4 nm.

Nanoemulsions 4 and 5 were mixed at a ratio of 1:1. The obtained transparent double nanoemulsion comprising a supersaturated solution of Coenzyme Q10 (ubiquinone) and tocopherol shows for at least 12 months no decoloration at 4° C., at room temperature, and at 37° C., and the original particle size of 63.1 nm remains stable. Thus, the double nanoemulsion has an excellent storage stability.

### Example 4

Transparent Double Nanoemulsion Comprising a Supersaturated Solution of Coenzyme Q10 and having a Very Small Droplet Size

Nanoemulsion 6—Composition

| | |
|---|---|
| Lecithin | 5% |
| Ubiquinone | 3% |
| Caprylic/Capric Triglyceride | 2% |
| Alcohol | 20% |
| Glycerin | 20% |
| Aqua | 50% |

Preparation of 1 kg of Nanoemulsion 6

Nanoemulsion 6 was prepared the same way as Nanoemulsion 3. A supersaturated stable nanoemulsion comprising Coenzyme Q10 was obtained. The particle size was 35.9 nm.

Nanoemulsion 7—Composition

| | |
|---|---|
| Lecithin | 5% |
| Vitamin E Acetate | 0.9% |
| Caprylic/Capric Triglyceride | 2% |
| Tocopherol | 0.1% |
| Alcohol | 20% |
| Glycerin | 20% |
| Aqua | 50% |

Preparation of 1 kg of Nanoemulsion 7

Nanoemulsion 7 was prepared the same way as Nanoemulsion 1. The particle size was 27.5 nm.

Nanoemulsions 6 and 7 were mixed at a ratio of 1:2. The obtained transparent double nanoemulsion comprising Coenzyme Q10 (ubiquinone) and tocopherol shows for at least 12 months no decoloration at 4° C., at room temperature and at 37° C., and the original particle size of 32.5 nm remains stable. Thus, the double nanoemulsion has an excellent storage stability.

### Example 5

Transparent Double Nanoemulsion having a High Content of Coenzyme Q10 as Supersaturated Solution

Nanoemulsion 8—Composition

| | |
|---|---|
| Lecithin | 5% |
| Ubiquinone | 8% |
| Vegetable Oil | 4% |
| Alcohol | 20% |
| Glycerin | 20% |
| Aqua | 43% |

Preparation of 1 kg of Nanoemulsion 8

50 g of lecithin were dissolved in 100 g of alcohol and added with stirring to 430 g of water and 200 g of glycerin. 80 g of Coenzyme Q10 (ubichinone) [CoQ10] were dissolved at 45° C. in 40 g of vegetable oil and 100 g of alcohol (ethanol). When this CoQ10 solution was again cooled to room temperature (25° C.) most of the CoQ10 recrystallized out from the solution after some hours. Therefore, the CoQ10 solution, having a temperature of 40° C., was added to the lecithin/alcohol/glycerin/water mixture and then homogenized six times at 1200 bar (1.2·10⁸ Pa) using a high pressure homogenizer of Microfluidics Corp (MT 110®). Thereby, the alcohol was homogeneously distributed in the nanoemulsion. An orange transparent nanoemulsion was obtained.

Determination of the particle size by means of photon correlation spectroscopy (Autosizer 3C®) shows a mean particle size of 38.7 nm.

Samples of this nanoemulsion can be incubated at 4° C., at room temperature, and at 37° C. for one year, without any essential change of the particle size. Measurement with a differential calorimeter (Perkin Elmer) does not show a phase transition for the Coenzyme Q10 in the nanoemulsion. This means that Coenzyme Q10 in the nanoemulsion is in dissolved form. Since at 4° C. and at room temperature the limit of solubility of Coenzyme Q10 in vegetable oil is clearly exceeded, the preparation in question is a stable nanoemulsion of a supersaturated solution.

Nanoemulsion 9—Composition

| | |
|---|---|
| Lecithin | 5% |
| Vegetable Oil | 4% |
| Tocopherol | 1% |
| Alcohol | 20% |
| Glycerin | 20% |
| Aqua | 50% |

Preparation of 1 kg of Nanoemulsion 9

Nanoemulsion 9 was prepared the same way as Nanoemulsion 1. A transparent nanoemulsion having a particle size of 33.6 nm was obtained.

Nanoemulsions 8 and 9 were mixed at a ratio of 3:1. The obtained transparent double nanoemulsion comprising Coenzyme Q10 (ubiquinone) and tocopherol shows for at least 12 months no decoloration at 4° C., at room temperature, and at 37° C., and the original particle size of 38.6 nm remains stable. Thus, the double nanoemulsion has an excellent storage stability.

1. A composition comprising at least two different nanoemulsions stabilized by lecithin each of which containing a liquid lipid, at least two of said lipids being incompatible with each other.

2. The composition of claim 1, wherein said composition is stable for at least six months without that the incompatible lipids react.

3. The composition of claim 2, wherein said composition is stable for at least two years without that the incompatible lipids react.

4. The composition of claim 1, wherein the particle size of the lipid droplets in all nanoemulsions is less than 80 nm, said nanoemulsion being transparent.

5. The composition of claim 4, wherein the particle size of the lipid droplets in all nanoemulsions is less than 45 nm.

6. The composition of claim 1, wherein said incompatible lipids are tocopherol and Coenzyme Q10.

7. The composition of claim 6, wherein the concentration of tocopherol is 0.1 to 20 percent by weight and the concentration of Coenzyme Q10 is 0.1 to 20 percent by weight.

8. The composition of claim 1, wherein at least one of the nanoemulsions comprises as liquid oil phase a solution of a lipophilic compound in oil, said solution being supersaturated at room temperature.

9. The composition of claim 8, wherein said supersaturated solution is stable for at least six months at 4° C.

10. The composition of claim 9, wherein said supersaturated solution is stable for at least three years at 4° C.

11. The composition of claim 8, wherein the lipophilic compound in said supersaturated solution is Coenzyme Q10.

12. The composition of claim 11, wherein the supersaturated nanoemulsion contains 0.1 to 20 percent of Coenzyme Q10.

13. A process for preparing a composition comprising at least two different nanoemulsions stabilized by lecithin each of which containing a liquid lipid, at least two of said lipids being incompatible, and at least one of said lipids being in solid form at room temperature, said process comprising the steps of:
dissolving in oil, separately from the other lipids, at least one of the incompatible solid lipids at a temperature at which it is soluble in said oil;
processing each of the solutions of lipids separately into an individual nanoemulsion by means of high pressure homogenization;
cooling each of said nanoemulsions down to room temperature thereby creating a stable supersaturated solution of said solid lipid; and;
combining the individual solutions to form said composition.

14. A process for preparing a composition comprising at least two different nanoemulsions stabilized by lecithin each of which containing a liquid lipid, at least two of said lipids being incompatible, and at least one of said lipids being in solid form at room temperature, said process comprising the steps of:
dissolving in a mixture of oil and an organic solvent, separately from the other lipids, at least one of the incompatible solid lipids at a temperature at which it is soluble in said mixture;
processing each of the solutions of lipids separately into an individual nanoemulsion by means of high pressure homogenization;
cooling each of said nanoemulsions down to room temperature thereby creating a stable supersaturated solution of said solid lipid; and;
combining the individual solutions to form said composition.

15. The process of claim 14, wherein said organic solvent is ethanol.

16. The use of a composition comprising at least two different nanoemulsions stabilized by lecithin each of which containing a liquid lipid, at least two of said lipids being incompatible to each other, in cosmetic preparations.

17. The use of a composition comprising at least two different nanoemulsions stabilized by lecithin each of which containing a liquid lipid, at least two of said lipids being incompatible with each other, in cell cultures.

18. The use of a composition comprising at least two different nanoemulsions stabilized by lecithin each of which containing a liquid lipid, at least two of said lipids being incompatible to each other, in nutrient compliments.
